(19) United States
(12) Patent Application Publication
(10) Pub. No.: US 2003/0216448 A1
(43) Pub. Date: Nov. 20, 2003

(54) Treatment of neuropathic pain

(76) Inventors: Lynne E. Rueter, Round Lake Beach, IL (US); Kathy L. Kohlhaas, Lake Zurich, IL (US)

Correspondence Address: WOOD, PHILLIPS, KATZ, CLARK & MORTIMER, Citicorp Center, Suite 3800, 500 West Madison Street, Chicago, IL, 60661-2511, US

(21) Appl. No.: 10/145,681

(22) Filed: May 15, 2002

(51) Int. Cl. A61K 31/4439; A61K 31/455

(52) U.S. Cl. 514/355; 514/343

(57) ABSTRACT

A method for treating a patient suffering from neuropathic pain, comprising administering to a patient in need of such treatment an effective amount of an agonist drug capable of binding to the neuronal nicotinic receptor (NNR) but which does not readily cross the blood-brain barrier.

### TECHNICAL FIELD

The present invention relates to a novel treatment of neuropathic pain. Specifically, the invention relates to a method for treating a patient suffering from neuropathic pain, including allodynia and to the reduction of side effects associated with agonist activation of central neuronal nicotinic receptors.

### BACKGROUND OF THE INVENTION

Pain is a sensation and a perception that is comprised of a complex series of mechanisms. In its most simple construction, it is a signal from the firing of nociception, touch and pressure receptors in the periphery that is transmitted to the spinal cord and finally to lower and higher centers of the brain. However, this signal can be modified in a multitude of ways at each level of the pain pathway. (See e.g Millan, M. J. (1999) The Induction of Pain: An Integrative Review, Progress in Neurobiology, 57, 1-164 (Pergamon Press) for an in depth review).

There are primarily three types of pain. Acute pain, termed nociception, is the instantaneous onset of a painful sensation in response to a noxious stimulus. It is considered to be adaptive because it can prevent an organism from damaging itself. For example, removing a hand from a hot stove as soon as pain is felt will prevent serious burns.

The second type of pain is persistent pain. Unlike acute pain, it usually has a delayed onset but can last for hours to days. It is predominately considered adaptive because the occurrence of persistent pain following injury can prevent further damage to the tissue. For example, the pain associated with a sprained ankle will prevent the patient from using the foot thereby preventing further trauma and aiding healing.

The final category of pain is chronic pain. It has a delayed onset and can last for months to years. In contrast to acute and persistent pain, chronic pain is considered maladaptive and is associated with conditions such as arthritis, nerve injury, AIDS and diabetes.

Chronic or neuropathic pain occurs in a variety of forms including spontaneous pain (painful sensation without an external stimulus), allodynia (painful sensation in response to a normally innocuous stimulus) and hyperalgesia (strong painful sensation to a mildly painful stimulus). It may be this diversity of symptoms that has made this condition difficult to treat clinically. In fact, current treatments are predominately the off label use of antidepressants and anticonvulsants. However, both antidepressants and anticonvulsants present problems for the patient.

Tricyclic antidepressants have the longest history of use in the treatment of neuropathic pain. Such drugs typically target the serotonergic and noradrenergic systems and increase the available extracellular levels of both serotonin and norepinephrine. It has been proposed that the postsynaptic activation of α2-adrenoceptors by norepinephrine may be the mechanism through which these compounds alleviate neuropathic pain. However, since antidepressants readily cross the blood-brain barrier, their ability to increase the levels of serotonin and norepinephrine may cause the undesired activation of other receptors leading to the high risk of centrally-mediated side effects. Side effects of antidepressants range from mild but irritating symptoms such as dry mouth and sedation to severe life threatening side effects such as postural hypotension and cardiac arrythmias. The elderly, who represent a large number of neuropathic patients, are particularly vulnerable to the more serious side effects of antidepressants.

The effectiveness of anticonvulsants in the treatment of various pain states, including neuropathic pain, has recently been evaluated (McQuay et al. (1995) Anticonvulsant Drugs For The Management of Pain: A Systematic Review, British Medical Journal 311, 1047-52). Similar to antidepressants, side effects frequently occur with these medications.

Due to the common occurrence of side effects with antidepressants and anticonvulsants and the limitations these place on the use of these compounds, there is a need for a treatment for neuropathic pain that does not rely on antidepressants or anticonvulsants. In addition, there is a need for a treatment for neuropathic pain that avoids centrally mediated side effects.

### SUMMARY OF THE INVENTION

In one aspect of the invention, a method of treating a patient suffering from neuropathic pain is provided by administering to a patient in need of such treatment an effective amount of an neuronal nicotinic receptor (NNR) agonist drug capable of binding to peripheral NNRs but which does not readily cross the blood-brain barrier.

In another aspect of the invention, a method of treating a patient suffering from allodynia is provided, said method comprising administering to a patient in need of such treatment an effective amount of an agonist drug capable of binding to at least one peripheral NNR but which does not readily cross the blood-brain barrier at doses for which an analgesic effect is observed.

In a still further aspect of the invention, a method of treating a patient suffering from neuropathic pain is provided, said method comprising administering to a patient in need of such treatment an effective amount of a quaternary nicotinic compound.

In yet another aspect of the invention, a method of treating a patient suffering from neuropathic pain by effecting peripheral NNRs at the level of the dorsal root ganglia (DRG) is provided by administering to a patient in need of such treatment an effective amount of a nicotinic agonist compound that does not readily cross the blood-brain barrier.

In yet another aspect ot the invention, a method of reducing side effects associated with the agonist activation of central NNRs is provided, said method comprising administering to a patient in need of such treatment an effective amount of a nicotinic agonist compound that does not readily cross the blood-brain barrier.

### DESCRIPTION OF THE DRAWINGS

The following is a description of the figures which are presented for the purposes of illustrating the invention and not for purposes of limiting the same.

FIG. 1 shows the effect of pretreatment of systemically administered chlorisondamine (0.4 μmol/kg, i.p. 30 minutes prior) on the antinociception induced by systemically administered A-85380 (0.75 μmol/kg, i.p.) as measured by the increase in paw withdrawal latency (mean±sem) in the rat paw withdrawal model of acute thermal pain using normal rats. Means represent the average of the left and right paw scores collapsed across the 15, 30 and 45 minute measures in order to show the main effect of dose. Five rats were used in each group. *p<0.05 vs. saline/saline.

FIG. 2A shows the effect of the pretreatment of i.c.v. (intracerebroventricular) chlorisondamine (10 μg 24 hours prior) on the analgesia induced by systemically administered A-85380 (0.75 μmol/kg, i.p.) as measured by the decrease in flinches (mean±sem) in the formalin model of persistent pain. Five rats were used in each group.

FIG. 2B shows the effect of pretreatment of systemically administered chlorisondamine (0.4 μmol/kg, i.p. 45-50 minutes prior) on the analgesia induced by systemically administered A-85380 (0.75 μmol/kg, i.p.) as measured by the decrease in flinches (mean±sem) in the formalin model of persistent pain. Flinches were measured over 20 minutes during phase 2. Six to seven rats were used in each group. *p<0.05 vs. saline/saline.

FIG. 3 shows the effects of multiple doses of the NNR agonist A-85380 on paw withdrawal threshold (mean±sem) in a test of mechanical allodynia in the spinal nerve ligation model of neuropathic pain. Paw withdrawal threshold (gram force) was determined using von Frey filament stimulation and the Dixon up-down method. Measures were taken before agonist injection (baseline) and at 15, 30, 60 and 120 minutes after agonist injection. Six rats were used at each dose.

FIG. 4 shows the effect of the pretreatment of systemically administered mecamylamine (1 μmol/kg, i.p. 30 minutes prior) on anti-allodynia induced by systemically administered A-85380 (0.75 μmol/kg, i.p.) as measured by the increase in paw withdrawal threshold (mean±sem) to mechanical stimulation in the rat spinal nerve ligation model of neuropathic pain. Means represent the withdrawal thresholds collapsed across the 15, 30 and 60 minute measures in order to show the main effect of treatment. Six rats were used in each group. *p<0.05 vs. saline/saline.

FIG. 5A shows the effect of the pretreatment of intracerebroventrical (i.c.v.) chlorisondamine (10 μg 24 hours prior) on anti-allodynia induced by systemically administered A-85380 (0.75 μmol/kg, i.p.) as measured by the increase in paw withdrawal threshold (mean sem) to mechanical stimulation in the rat spinal nerve ligation model of neuropathic pain.

FIG. 5B shows the effect of the pretreatment of peripherally administered chlorisondamine (0.4 /μmol/kg, i.p. 30 minutes prior) on the anti-allodynia induced by systemically administered A-85380 (0.75 μmol/kg, i.p.) as measured by the increase in paw withdrawal threshold (mean±sem) to mechanical stimulation in the rat spinal nerve ligation model of neuropathic pain. Means represent the withdrawal thresholds collapsed across the 15, 30 and 60 minute measures in order to show the main effect of treatment. Four to six rats were used in each group. *p<0.05 vs. saline/saline.

FIG. 6 shows the effect of the pretreatment of systemically administered hexamethonium (19 μmol/kg, i.p. 30 minutes prior) on the anti-allodynia induced by systemically administered A-85380 (0.75 μmol/kg, i.p.) as measured by the increase in paw withdrawal threshold (mean±sem) to mechanical stimulation in the rat spinal nerve ligation model of neuropathic pain. Means represent the withdrawal thresholds collapsed across the 15, 30 and 60 minute measures in order to show the main effect of treatment. Five rats were used in each group. *p<0.05 vs. saline/saline.

FIG. 7 shows the effect of pretreatment of systemically administered chlorisondamine (0.4 μmol/kg, i.p. 30 minutes prior) on the antinociception induced by systemically administered A-85380 (0.75 μmol/kg, i.p.) as measured by the increase in paw withdrawal latency (mean±sem) in the rat paw withdrawal model of acute thermal pain using neuropathic rats. Means represent the average of the left and right paw scores collapsed across the 15, 30 and 45 minute measures in order to show the main effect of treatment. Six rats were used in each group. *p<0.05 vs. saline/saline.

FIG. 8A shows the effect of multiple doses of A-85380 injected into the plantar surface of the hind paw on the affected (ipsalateral) side on the paw withdrawal threshold (mean±sem) to mechanical stimulation of the neuropathic paw in the rat spinal nerve ligation model of neuropathic pain.

FIG. 8B shows the effect of multiple doses of A-85380 injected into the plantar surface of the hind paw on the unaffected (contralateral) side on the paw withdrawal threshold (mean±sem) to mechanical stimulation of the neuropathic paw in the rat spinal nerve ligation model of neuropathic pain. Measures were taken before agonist injection (baseline) and at 15, 30, 60 and 120 minutes after agonist injection. Six rats were used at each dose/paw combination. *p<0.05 vs. saline at same time point.

FIG. 9A shows the effect of different doses of A-85380 infused onto the L5 DRG on the paw withdrawal threshold (mean±sem) to mechanical stimulation in the rat spinal nerve ligation model of neuropathic pain. Measures were taken before agonist injection (baseline) and at 15, 30, and 60 minutes after agonist injection. Six rats were used at each dose/paw combination. *p<0.05 vs. saline at same time point.

FIG. 9B shows the effect of the pretreatment of systemically administered chlorisondamine (0.4 μmol/kg, i.p. 30 minutes prior) on the anti-allodynia induced by A-85380 infused onto the DRG (20 μg) as measured by the increase in paw withdrawal threshold (mean±sem) to mechanical stimulation in the rat spinal nerve ligation model of neuropathic pain. Means represent the withdrawal thresholds collapsed across the 15, 30 and 60 minute measures in order to show the main effect of treatment. Six rats were used in each group. *p<0.05 vs. saline/saline.

FIG. 10 shows the comparison of the effects of A-85380 administered either i.p., i.d. into the affected paw or onto the L5 DRG on the paw withdrawal threshold (mean±sem) to mechanical stimulation in the rat spinal nerve ligation model of neuropathic pain. The doses of A-85380 administered i.d. or DRG were converted into systemic doses based on the body weight of each rat and graphed as the mean equivalent to systemic dose. Paw withdrawal threshold means represent the fifteen minute time point only.

### DETAILED DESCRIPTION

The term used to describe pain reduction is analgesia (antinociception, antiallodynia), which may be described as the reduction of pain and, from the perspective of this invention, the reduction of pain associated with neuropathic pain, as well as allodynia often associated with neuropathic pain.

The term “agonist” is defined as a compound that exhibits 30% or greater efficacy in a functional assay in comparison to the endogenous ligand acetylcholine or the exogenous ligand nicotine.

The terms “substantially unable to cross the blood brain barrier” and “not readily able to cross the blood brain barrier” refer to the inability of compounds to cross the blood-brain barrier and activate central NNRs at doses that are fully able to activate NNRs in the periphery.

This invention provides a method of treating neuropathic pain in a patient requiring such treatment while minimizing the risk of centrally mediated side effects in the patient. The inventors have made the surprising discovery that neuropathic pain can be ameliorated, at least in part, by agonist binding to the peripheral NNR, wherein the agonist drug is substantially unable to penetrate the blood-brain barrier thereby avoiding inducing centrally mediated central nervous system (CNS) side effects. The inventors are the first to demonstrate a substantial role for peripheral NNRs in neuropathic pain often associated with allodynia. Any suitable NNR agonist unable to readily cross the blood-brain barrier may be utilized in the method of the present invention.

The inventors have made the surprising discovery that agonistic binding to peripheral NNRs provides relief from allodynia absent agonist binding to centrally located NNRs. This new discovery provides a novel method of treating a patient who needs relief from allodynia by administering to the patient an NNR agonist that is substantially unable to cross the blood-brain barrier. This new method of treating allodynia dramatically reduces the potential for centrally mediated CNS side effects. Thus, the new method of treatment is of particular value to a patient requiring such treatment for allodynia without incurring centrally mediated side effects.

All references contained herein are fully incorporated by reference.

Methods

Animals

Male Sprague Dawley rats (80-100 g for nerve ligation alone or 200-225 g for nerve ligation plus i.c.v. cannulation or DRG catheterization) were purchased from Charles River (Portage, Mich.). Prior to surgery, animals were group-housed and maintained in a temperature regulated environment (lights on between 7:00 a.m. and 8:00 p.m.). Following nerve ligation surgery alone, animals were group housed. Two weeks after surgery, experimentation began. Animals were between 250-350 g during the experiments. Following nerve ligation surgery plus either i.c.v. cannulation or DRG catheterization, animals were individually housed. Experimentation began one week following surgery when animals were between 250-350 g. Rats had access to food and water ad libitum.

Surgical Procedures

Under halothane anesthesia, the L5 and L6 spinal nerves were tightly ligated in the manner described previously by Chung et al. (Kim S. H., Chung J. M., An experimental model for peripheral neuropathy produced by segmental spinal nerve ligation in the rat. Pain 1992; 50:355-363.). Briefly, an incision was made on the dorsal portion of the hip, and the muscle was blunt dissected to reveal the spinal processes. The L6 transverse process was removed, and the left L5 and L6 spinal nerves were tightly ligated with 5.0 braided silk suture. The wound was cleaned, the membrane sewn with 4.0 dissolvable Vicryl suture and the skin closed with wound clips.

Implantation of the DRG catheters was performed immediately following nerve ligation, with animals maintained under halothane for the entire procedure (35-40 minutes). Catheters were constructed of PE 20 with small pieces of tygon tubing cemented along the catheter to allow for suture anchoring points during surgery. The left L5 DRG was exposed by removing the posterior articular process of the L5 vertebra. Gelfoam was inserted into the cavity to prevent the catheter from damaging the ganglion. The catheter was sutured to the muscle and fascia, then run subcutaneously to exteriorize between the shoulder blades. Saline was infused into the catheter, and it was heat sealed.

The implantation of i.c.v. cannulae were conducted under sodium pentobarbital (50 mg/kg, i.p. Nembutal) anesthesia with nerve ligation occurring just after the implantation; animals were maintained under anesthesia for the entire procedure (40-45 minutes). Animals were implanted with a 22 gauge guide cannulae (Plastics One, Roanoke Va.) cut to 6 mm. The stereotaxic coordinates from bregma were 1.0 mm posterior, 1.6 mm lateral, and 4.5 ventral (Paxinos G. and Watson C., The rat brain in stereotaxic coordinates. New York: Academic Press, 1997). Three skull screws were attached for stability, and the implant was covered with cranioplastic acrylic.

Infusion and Injection Procedures

For i.c.v. (intracerebroventricular) infusion, chlorisondamine (10 μg) was dissolved in 5 μl phosphate buffered saline and infused by syringe pump at a rate of 5 μl/min. The infusion was performed 24 hours prior to testing in order to reduce stress to the animals immediately prior to behavioral testing.

For all other routes of administration including intraperotineal (i.p.), intradermal (i.d.) or onto the DRG, chlorisondamine was dissolved in saline. The final volumes for i.p. injections were 1 ml/kg, for i.d. were 50 μl and for DRG injections were 10 μl. All injections were made by hand held syringe.

Behavioral Assessment

For the assessment of acute pain, a paw thermal stimulator was used to evaluate nociceptive responses to an acute thermal stimulus (Anesthesiology Research Laboratory, Department of Anesthesiology, University of California at San Diego, La Jolla, Calif.). The device and behavioral procedure have been described previously (Hargreaves et al., A new and sensitive method for measuring thermal nociception in cutaneous hyperalgesia, Pain 1988, 32:77-88; Dirig et al., Characterization of variables defining hind paw withdrawal latency evoked by radiant thermal stimuli, J. Neurosci. Meth. 1997; 76:183-191). Briefly, rats were placed in Plexiglas boxes on a glass surface maintained at 30° C. and allowed to acclimate for 30 minutes. The measured nociceptive response was the latency to withdraw the hind paw when heated by light from a focused projector lamp (current set at 4.8 A). The final score was the mean of the latencies for the left and right paw. To avoid tissue damage, the maximal response latency prior to automatic shutoff of the lamp was 20.5 sec.

The formalin test was used as an assessment of persistent pain. The method, described previously (Bannon et al. 1998) is briefly as follows. Following acclimation to the test environment, 50 μl of a 5% formalin solution was injected subcutaneously into the dorsal surface of one of the rear paws. Only phase two, defined as the 20 minute period from 30 to 50 minutes post formalin injection, was scored for nocifensive behaviors including flinching, licking, and biting.

For the assessment of neuropathic pain, mechanical allodynia in the affected paw of animals who had undergone spinal nerve ligation was evaluated using von Frey filaments. As described previously (Chaplan et al., Quantitative assessment of tactile allodynia in the rat paw. J Neurosci Meth, 1994; 53:55-62), two weeks following surgery, rats were acclimated to the testing box which was constructed of plexiglass with a wire mesh floor to allow access to the planter surface of the hind paws. Using the Dixons Up-Down method, a baseline level of allodynia was taken with allodynia defined as a withdrawal threshold ≦4 g. Test compounds were then administered and subsequent withdrawal thresholds determined.

Experimental Timelines

For acute thermal pain experiments, a baseline withdrawal threshold was determined, and then chlorisondamine or its vehicle was administered. Following 30 minutes, A-85380 or its vehicle was administered and measures were taken at 15, 30 and 45 minutes post agonist injection.

For formalin experiments, chlorisondamine or its vehicle was infused either 24 h or 40-50 minutes prior to further manipulation. A-85380 was then injected and, 5 minutes later, formalin was injected into the paw. Measures were taken between 30 and 50 minutes post formalin injection.

For spinal nerve ligation experiments, the timing of antagonist administration depended upon the route of administration. With i.c.v. administration, chlorisondamine or its vehicle was given 24 hours prior to further manipulation. On the day of the experiment, a baseline allodynia score was determined and then A-85380 or its vehicle control was given and measures taken at 15, 30 and 60 minutes post agonist injection. Dose response curves for i.p., i.d. and DRG administration were also determined using this time line. In contrast, with i.p. administration of an antagonist, the baseline allodynia was determined first, the antagonist or its vehicle was injected and then, 30 minutes later, A-85380 or its vehicle was injected and measures were taken at 15, 30 and 60 minutes post agonist injection.

Compounds

The following compounds were dissolved in their appropriate vehicles for i.c.v., i.p., i.d. or DRG injections: A-85380 (i.e. 3-(2(S)-azetidinylmethoxy)pyridine dihydrochloride (available from RBI, Natick, Mass.; Sullivan et al, 1996); chlorisondamine diiodide (available from Tocris, Ballwin, Mo.; Clarke et al, 1994); mecamylamine hydrochloride (Sigma; St. Louis, Mo.); and hexamethonium (Sigma).

Statistical Analyses

For experiments with repeated measures, i.e. acute thermal pain and mechanical allodynia, data were first analyzed using a two way repeated measures analysis of variance (ANOVA) with two independent factors. For experiments without repeated measures, i.e. formalin, data were first analyzed using a two way ANOVA with two independent factors. If there was a significant interaction of both factors or of both factors and time, subsequent post hoc one way ANOVAs were performed using each treatment combination as an independent group. The difference in paw withdrawal latencies between the affected and unaffected paw of neuropathic animals tested in acute thermal pain was assessed using a paired t-test. All post hoc significance was determined using Fishers LSD.

The use of preclinical models to study pain has allowed a more in depth investigation of differing pain states including the determination of the multiple sites and mechanisms of action for different types of pain. For example, studies indicate that acute pain and persistent pain are influenced by tonic descending inhibitory components that limit the transmission of pain at the level of the spinal cord (Proudfit H K, and Hammond D L., Alterations in nociceptive threshold and morphine-induced analgesia produced by intrathecally administered amine antagonists. Brain Res 1981, 218:393-399; Omote et al., 1998; Kaneko M, Hammond I D, Role of spinal γ-aminobutyric acid_{A} receptors in formalin-induced nociception in the rat. J Pharmacol Exp Ther 1997, 282:928-938).

In contrast, in neuropathic pain, while similar descending inhibitory influences may limit the expression of mechanical allodynia (Xu et al., Endogenous noradrenergic tone controls symptoms of allodynia in the spinal nerve ligation model of neuropathic pain, Eur J Pharmacol 1999, 366:41-45), investigators have identified an important descending tonic facilitation of tactile allodynia as well as evidence for an additional ascending facilitation (Ossipov et al., Mediation of spinal nerve injury induced tactile allodynia by descending facilitatory pathways in the dorsolateral funiculus in rats, Neurosci Lett 2000, 290:129-132; Sung et al., Supraspinal involvement in the production of mechanical allodynia by spinal injury in rats, Neurosci Lett 1998, 246:117-119; Sheen K. and Chung J M. Signs of neuropathic pain depend on signal from injured nerve fibers in a rat model, Brain Res 1993, 610:62-68). In demonstration of further differences in pain states, the descending facilitation required for the expression of mechanical allodynia does not appear to play a role in thermal hyperalgesia caused by spinal nerve ligation (Bian et al., Tactile allodynia, but not thermal hyperalgesia, of the hind limbs is blocked by spinal transection in rats with nerve injury, Neurosci Lett 1998, 241:79-82; Kauppila et al., Influence of spinalization on spinal withdrawal reflex responses varies depending on the submodality of the test stimulus and the experimental pathophysiological condition in the rat, Brain Res 1998, 797:234-242).

The neuronal mechanisms that underlie the antinociception induced by NNR agonists in acute pain models have also been shown to differ based upon the type of acute pain elicited or upon the specific NNR agonist used (Curzon et al., Differences between the antinociceptive effects of the cholinergic channel activators A-85380 and (±)-epibatidine in rats, J Pharmacol Exp Ther 1998; 287:847-853). Since NNR agonists also have been shown to be effective in persistent and neuropathic pain models (Bannon et al., Broad-spectrum, non-opioid analgesic activity by selective modulation of neuronal nicotinic acetylcholine receptors. Science 1998, 279:77-81; Kesingland et al., Analgesia profile of the nicotinic acetylcholine receptor agonist, (+)-epibatidine and ABT-594 in models of persistent inflammatory and neuropathic pain, Pain 2000, 86:113-118) it was an objective of the present study to further delineate the possible different neuronal pathways that underline NNR-agonist-induced antinociception, analgesia and anti-allodynia. The NNR agonist A-85380 was used throughout in order to facilitate comparison amongst the various pain models.

### EXAMPLE 1

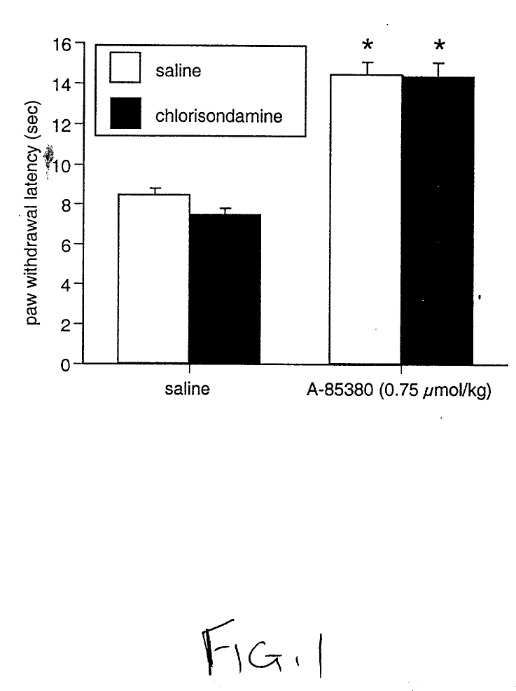

The effect of systemically administered chlorisondamine in a model of acute thermal pain was examined. Chlorisondamine, a quaternary NNR quasi-irreversible antagonist that does not readily cross the blood-brain barrier was used to identify the central and peripheral actions of A-85380. The effect of systemically administered chlorisondamine (0.4 μmol/kg, i.p.) on A-85380 (0.75 μmol/kg, i.p.) was assessed. As shown in FIG. 1, i.p. chlorisondamine had no effect on the antinociceptive action of A-85380 in acute thermal pain (interaction of agonist and antagonist, p=0.42, effect of antagonist, p=0.35).

### EXAMPLE 2

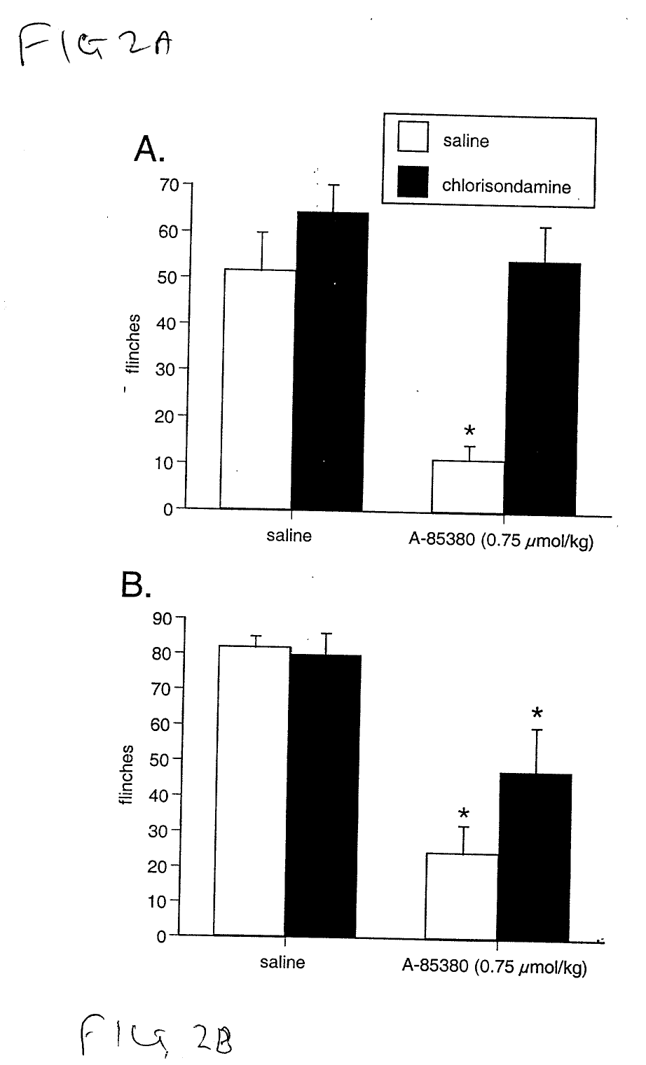

The effect of centrally and systemically administered chlorisondamine in a model of persistent pain was examined. Using phase two of the formalin model of persistent pain, chlorisondamine (10 μg) was administered i.c.v. 24 hours prior to the systemic administration of A-85380. The i.c.v. administered chlorisondamine completely blocked the analgesic effects of systemically administered A-85380 (0.75 μmol/kg, i.p.; interaction of antagonist and agonist, p=0.048; effect of antagonist/agonist treatment combination p=0.0002; FIG. 2A). In contrast, when chlorisondamine was administered systemically 40-50 minutes prior to the formalin injection, there was no significant attenuation in A-85380-induced analgesia in phase two of the formalin test (interaction of agonist and antagonist, p=0.12, effect of antagonist p=0.19; FIG. 2B).

### EXAMPLE 3

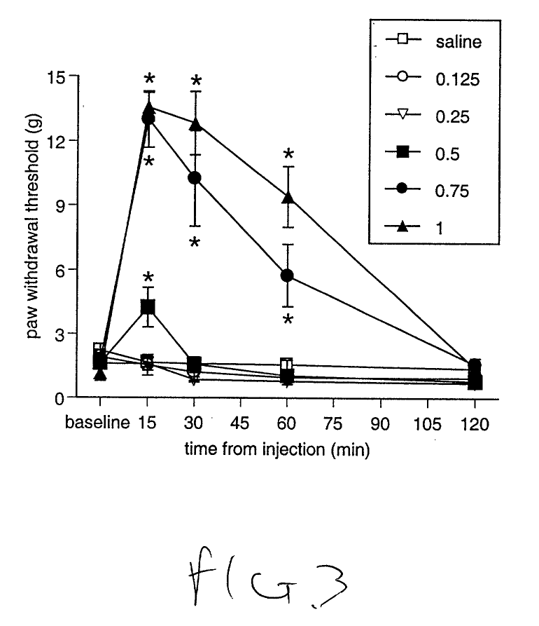

The effect of the NNR Agonist A-85380 in a model of neuropathic pain was examined. Systemically administered A-85380 induced a dose dependent anti-allodynia in rats with neuropathy secondary to the tight ligation of spinal nerves L5 and L6 (interaction of dose and time, p<0.0001, effects of A-85380 at 15, 30 and 60 minutes, p<0.0001), see FIG. 3. A-85380 at 0.5-1.0 μmol/kg, i.p. induced behaviors such as prostration and ataxia immediately following injection. However, these effects abated by the 15 minute time point and did not interfere with behavioral testing.

### EXAMPLE 4

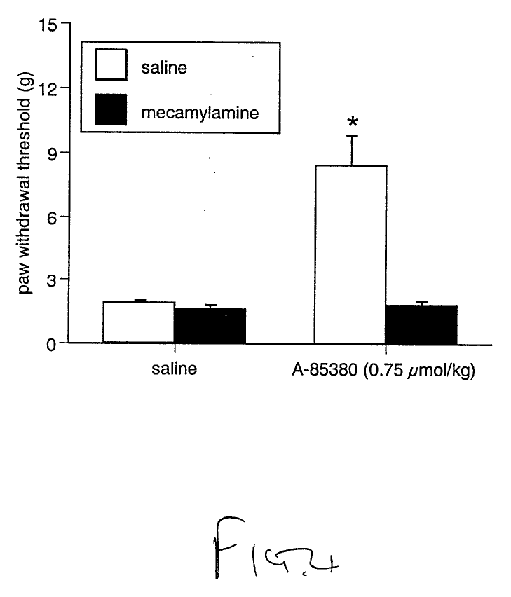

The effect of centrally and systemically administered chlorisondamine NNR antagonists on A-85380 induced anti-allodynia was examined. To determine if the anti-allodynic action of A-85380 was mediated by NNRs, the ability of the NNR channel blocker mecamylamine to block A-85380 induced anti-allodynia was assessed. Mecamylamine, 1 μmol/kg, i.p. given 30 minutes prior to A-85380, completely blocked the anti-allodynia of A-85380 (interaction of antagonist and agonist, p<0.0001; effect of antagonist/agonist treatment combination p<0.0001: FIG. 4). It also blocked the prostration and ataxia that immediately followed injection of the agonist.

### EXAMPLE 5

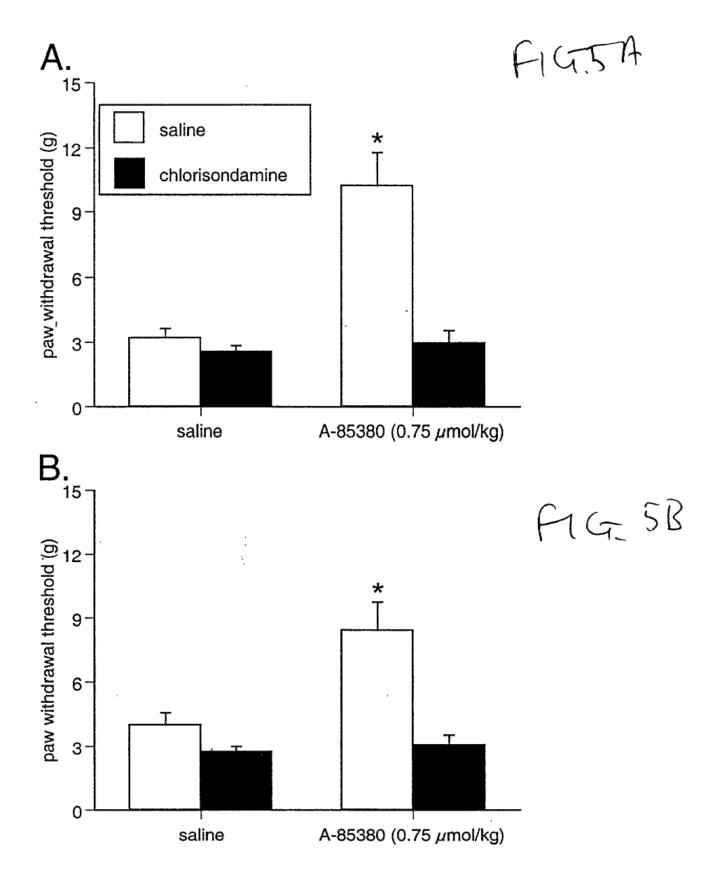

The site of action of A-85380-induced anti-allodynia was examined. To determine the site of action of A-85380-induced anti-allodynia, the quaternary NNR antagonist chlorisondamine was given either i.c.v. or i.p. prior to A-85380 administration. When given i.c.v. 24 hours prior to behavioral testing, chlorisondamine (10 μg) completely blocked the anti-allodynia induced by systemically administered A-85380 (0.75 μmol/kg, i.p.; interaction of agonist and antagonist, p=0.01; effect of antagonist/agonist treatment combination p=0.0004; FIG. 5A). Surprisingly, in contrast to the lack of significant effects of systemically administered chlorisondamine in acute and persistent pain models, systemically administered chlorisondamine (0.4 μmol/kg, i.p.) completely blocked the anti-allodynic effects of A-85380 (0.75 μmol/kg, i.p.) in a model of neuropathic pain (interaction of antagonist and agonist, p=0.04, effect of antagonist/agonist treatment combination, p=0.0006; FIG. 5B).

### EXAMPLE 6

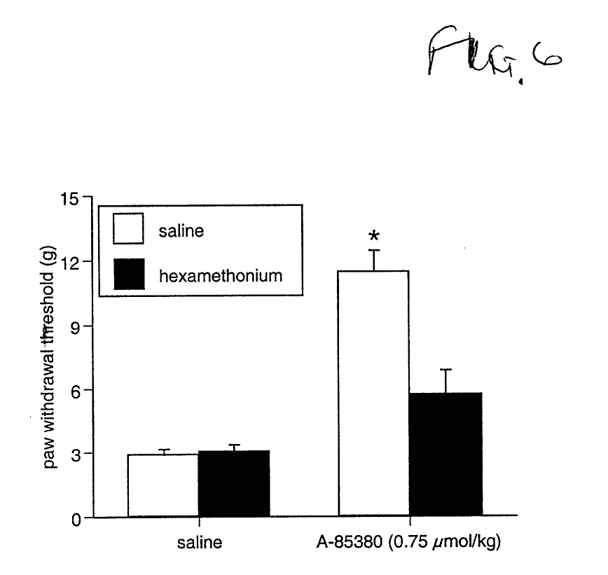

Verification of a systemic site of anti-allodynic action of A-85380 was examined. In order to verify the ability of systemically administered chlorisondamine to block the anti-allodynic effect of A-85380 in the spinal nerve ligation model of neuropathic pain, the effect of another quaternary NNR antagonist, hexamethonium, was assessed. Hexamethonium, 19 μmol/kg, i.p. given 30 minutes prior to agonist administration, also blocked the anti-allodynic effect of systemically administered A-85380 (0.75 μmol/kg, i.p.; interaction of antagonist and agonist, p=0.02, effect of antagonist/agonist treatment combination, p=0.0003, FIG. 6).

### EXAMPLE 7

Due to the proximity of the spinal nerve ligation to the spinal cord and blood-brain barrier (“BBB”), there was a concern that the ability of peripherally administered chlorisondamine to block A-85380 induced anti-allodynia was in fact due to the penetration of chlorisondamine into the CNS through a permanent weakness in the BBB incidentally caused during surgery.

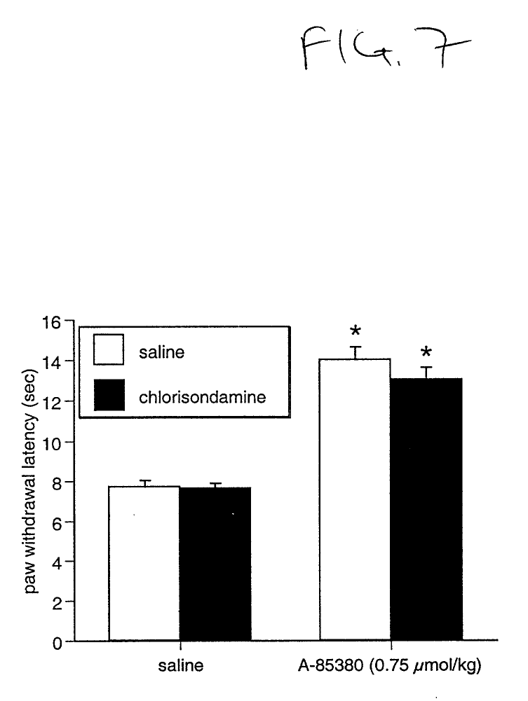

To address this concern, neuropathic rats were assessed in the acute thermal model of paw withdrawal. As demonstrated above, peripherally administered chlorisondamine is completely ineffective in antagonizing A-85380 induced antinociception indicating a completely central. site of action of the agonist in this pain model. If chlorisondamine were crossing the BBB in neuropathic animals, peripherally administered chlorisondamine in these animals should partially or fully block the antinociceptive effects of A-85380. This was not the case as shown in FIG. 7. Systemically administered chlorisondamine (0.4 μmol/kg/, i.p.) did not alter the antinociception induced by systemically administered A-85380 (0.75 μmol/kg, i.p.) when using neuropathic rats in the paw withdrawal model of acute thermal pain (interaction of antagonist and agonist, p=0.46, effect of antagonist p=0.41). Interestingly, in the baseline measurement only, there was a small but significantly lower paw withdrawal threshold for the affected paw (7.2±0.3 s) vs. the unaffected paw (8.5±0.3 s; p=0.003; data not shown).

### EXAMPLE 8

The site of action for the peripheral effects of A-85380 in a model of neuropathic pain was examined. The ability of A-85380 to induce an anti-allodynic action when applied directly to the primary receptive field was assessed by injecting A-85380 into the plantar surface of either the affected (ipsalateral to the ligation) or unaffected (contralateral to the ligation) paw and then measuring the degree of allodynia in the affected paw. A-85380, in doses of 10, 20 and 50 μg/rat, i.d. induced changes in paw withdrawal threshold that were dependent upon time, paw and dose (interaction, p=0.01).

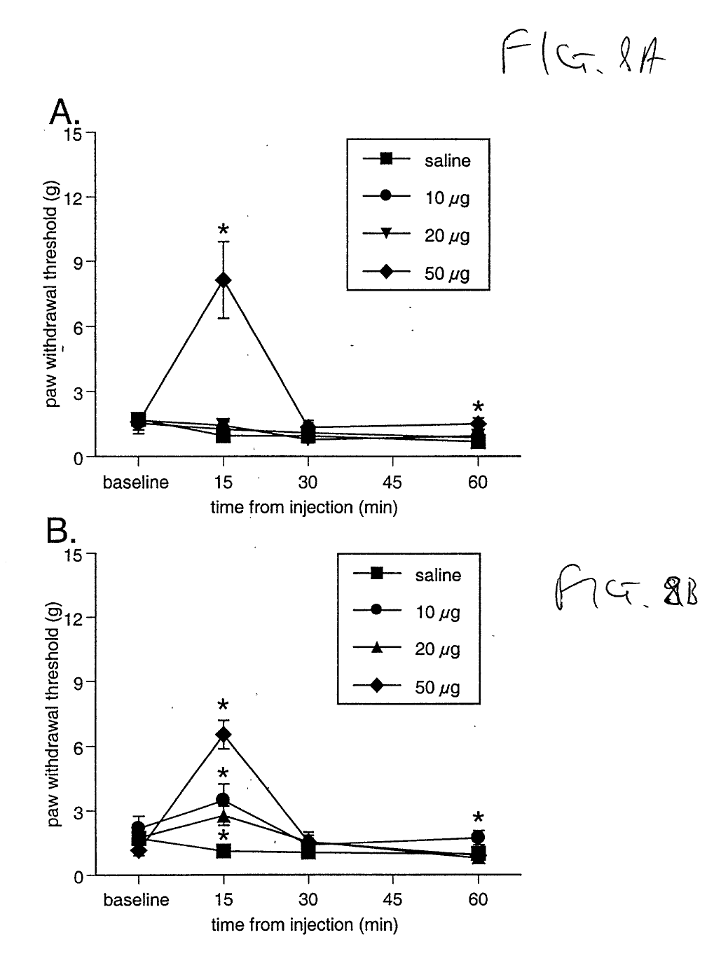

Analyses were therefore made of each paw individually. For the ipsalateral paw injection, only the 50 μg dose of A-85380 had an anti-allodynic effect, predominately at the 15 minute time point although the 60 minute time point was also significantly different from saline (interaction of time and dose, p<0.0001, effect of dose at 15 min, p<0.0001, at 30 min, p=0.47, at 60 min, p=0.005, FIG. 8A). For the contralateral paw injection, all three doses of A-85380 induced a significant anti-allodynia, again the predominate effect being at the 15 minute time with a very small but significant effect of 10 μg only at 60 minutes (interaction of time and dose, p<0.0001, effect of dose at 15 min p<0.0001, at 30 min, p=0.36, at 60 min, p=0.03, FIG. 8B).

### EXAMPLE 9

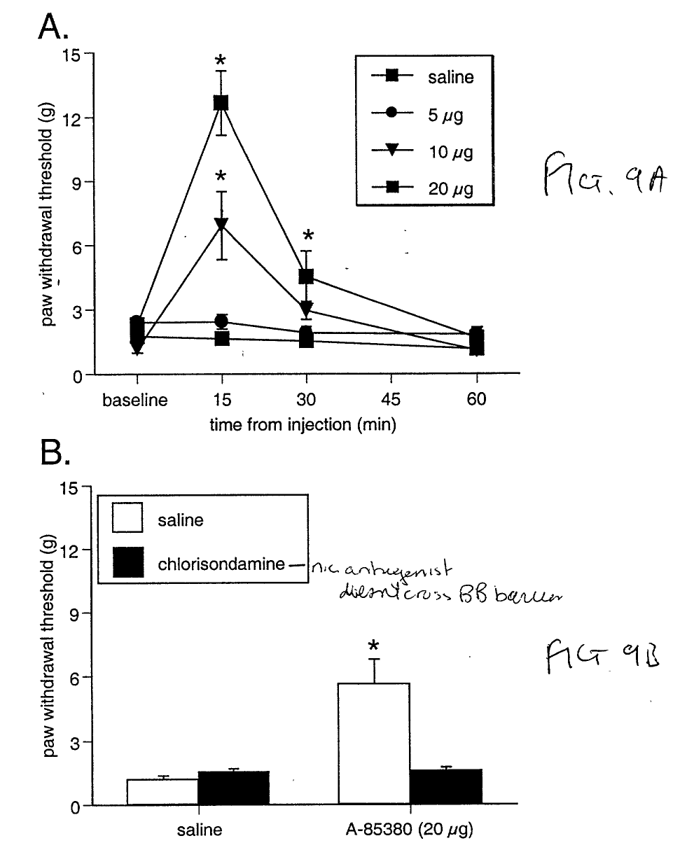

The ability of A-85380 to induce an anti-allodynic action when applied directly onto the DRG was examined. A-85380 was fused onto the L5 DRG on the side of the ligation. Both 10 and 20 μg of A-85380 infused onto the DRG induced a significant anti-allodynia which lasted 15 and 30 minutes, respectively (interaction of time and dose, p<0.0001, effect of dose at 15 min, p<0.0001, at 30 min, p=0.007, at 60 min, p=0.33, FIG. 9A). Pretreatment with chlorisondamine, 0.4 μmol/kg, i.p. 30 minutes prior, completely blocked the anti-allodynia induced by 20 μg A-85380 infused onto the DRG (p<0.0001; FIG. 9B).

### EXAMPLE 10

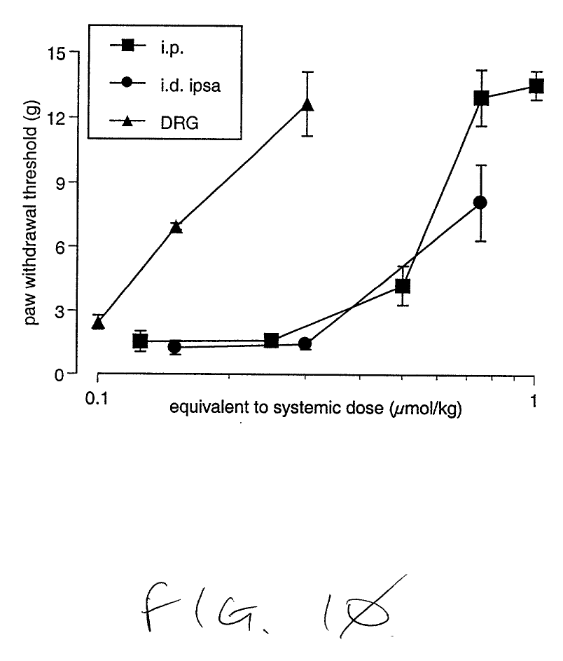

A comparison was then made between i.p., i.d., and DRG administration of A-85380. In order to allow a more direct comparison, the i.d. and DRG doses were first converted to their equivalent systemic dose, μmol/kg, by determining the dose received by each rat according to his body weight and then calculating the mean dose given. The fifteen-minute time point of the i.p., ipsalateral i.d., and DRG routes of administration are shown in FIG. 10. While the potency to increase the paw withdrawal threshold is similar between i.p. and i.d. administration, A-85380 is decidedly more potent when administered directly onto the DRG.

Discussion

The site(s) of action that underlie the reduction in pain responsiveness induced by the NNR agonist A-85380 differ according to the type of pain model used. Chlorisondamine, a quaternary NNR quasi-irreversible antagonist that does not readily cross the blood brain barrier was used to separate central and peripheral actions of A-85380 (Clarke et al., The pharmacology of the nicotinic antagonist, chlorisondamine, investigated in rat brain and autonomic ganglion. Br J Pharmacol 1994, 111:397-405). For acute thermal pain, studies with systemically administered vs. centrally administered chlorisondamine indicated that the site of antinociceptive is mediated centrally (see Curzon et al., Differences between the antinociceptive effects of the cholinergic channel activators A-85380 and (±)-epibatidine in rats, J Pharmacol Exp Ther 1998, 287:847-853; also see FIG. 1).

Similar to acute thermal pain, the analgesic action of A-85380 in a persistent pain model was also shown to be mediated predominately in the CNS, as indicated by the ability of centrally administered chlorisondamine to block A-85380 induced analgesia while peripheral administration of the antagonist had no significant effect (FIG. 2).

In contrast to the acute thermal and persistent pain model, the present study demonstrated a substantial role for peripheral NNRs in the anti-allodynia induced by the NNR agonist A-85380. A-85380 induced a dose-dependent anti-allodynia in the spinal nerve ligation model of neuropathic pain, an effect mediated by NNRs as demonstrated by the full blockade of the anti-allodynia with the NNR antagonist mecamylamine (FIGS. 3 and 4). Furthermore, the ability of both centrally and peripherally administered chlorisondamine to antagonize the anti-allodynia induced by A-85380 demonstrated that there is both a central and peripheral site of the anti-allodynic action (FIG. 5).

Because the peripheral site of anti-allodynic action for a NNR agonist is a novel finding, it was deemed necessary to verify this result. Thus, the peripheral antagonism of A-85380 was also demonstrated using the NNR antagonist hexamethonium (FIG. 6). In addition, a defect in the blood-brain barrier caused by the ligation surgery was ruled out by the finding that peripherally administered chlorisondamine does not alter antinociception in the paw withdrawal assay of acute thermal pain in neuropathic rats (FIG. 7).

With verification of the peripheral action of A-85380 established, the exact location of peripheral anti-allodynia was investigated. Injection of A-85380 directly into the primary receptive field of the sensory neurons in the plantar surface of the ipsalateral paw induced a small but significant anti-allodynia. However, equivalent or greater anti-allodynia was achieved when A-85380 was injected into the contralateral paw, indicating both effects were most likely due to a systemic effect of A-85380, not a local effect at the primary receptive field (FIG. 8).

In contrast to the lack of a selective effect of A-85380 at the primary receptive field, A-85380 induced a dose-dependent anti-allodynic effect when infused directly onto the L5 DRG (FIG. 9). This effect of A-85380 was blocked by the pretreatment with systemically administered chlorisondamine indicating that it is NNR-mediated. A comparison of the effects of A-85380 when administered systemically, into the primary receptive field or onto the DRG show that A-85380 is most potent when infused onto the DRG, thus suggesting that this may be an important peripheral site of action for NNR agonist-induced anti-allodynia in a rat model of neuropathic pain (FIG. 10).

While there appears to be a good deal of similarity between antinociception and analgesia, in terms of the mechanisms of action delineated in preclinical studies involving NNR ligands as well as other receptor ligands and in terms of the therapeutic pharmacology found in the clinic, studies indicate that anti-allodynia in neuropathic pain is quite distinct (Curzon, et al, J. Pharmacol Exp Ther 1998; 287:847-853; Rueter et al, Brain Res. 2000; 872:93-101; Rogers and Iwamoto, J. Pharmacol Exp Ther 1993; 267:341-349; Omote et al, Brain Res. 1998; 814:194-198; Sugimoto et al, Neuropharmacology 1986; 25:481-485; Kaneko and Hammond, J. Pharmacol Exp Ther 1997; 282:928-938; Millan, Prog. Neurobiol 1999; 57:1-164). Specifically, neuropathic pain clearly alters the functioning of both peripheral and central processes such as inducing ectopic discharge in the sensory neurons and inducing central sensitization, respectively (Gold, Pain 2000; 84:117-120; Attal and Bouhassira, Acta Neurol Scand 1999; Suppl. 173:12-24; Sheen and Chung, Brain Res. 1993; 610:62-68; Sung et al, Neurosci Lett 1998; 246:117-119; Bian et al, Neurosci Lett 1998; 241:79-82; Ossipov et al, Neurosci Lett 2000; 290:129-132; Lee et al, Neurosci Lett 2000; 290:129-132). Therefore, theoretically, inhibition of pain transmission and the subsequent relief of mechanical allodynia could occur either through actions of a therapeutic agent in the periphery or in the CNS.

The differential effects of site injections found in the present study suggest that there is a peripheral site of anti-allodynic action mediated by NNR in the DRG but not the primary receptive field (FIGS. 8, 9, 10). The present study is the first to report the ability of a NNR agonist to reduce neuropathic pain at the level the DRG and, indeed, the level of pain relief following NNR agonist infusion at the DRG is much greater than that of any compound of any other class reported previously (FIG. 9; Liu et al., 2000, Pain 2000; 85:503-521; Lyu et al., Brain Res. 2000; 871:98-103). Most NNR subunits have been reported to exist in the DRG including, but not limited to, α₇, α₃, and α₄ (Ninkovic and Hunt, Brain Res. 1983; 272:57-60; Boyd et al., 1991, J. Neurobiol 1991; 22:1-14). Thus, NNRs are in a position to alter pain transmission at the level of the DRG, and the present study suggests they may play an important role in reducing neuropathic pain transmission at this site.

### EXAMPLE 11

A-420503 (IR, 5S) 5-(3,6-diazabicyclo [3.2.0]hept-6-yl)-N-hydroxy-nicotinamide) infuses significant anti-allodynia at 500 mg which is equivalent to 4.77 μmol/kg, i.p. The effects at the DRG were blocked by pretreatment with an NMR antagonist. When injected i.p., it induces significant anti-allodynia at 62 μmol/kg, without any notable side effects.

While the invention is described above in connection with preferred or illustrative embodiments, these embodiments are not intended to be exhaustive or limiting of the invention. Rather, the invention is intended to cover all alternatives, modifications and equivalents included within its spirit and scope of the invention, as defined by the appended claims.

What is claimed:

1. A method for treating a patient suffering from neuropathic pain, comprising administering to a patient in need of such treatment an effective amount of an agonist drug capable of binding to peripheral neuronal nicotinic receptors but which does not readily cross the blood-brain barrier.

2. The method for treating a patient suffering from neuropathic pain according to claim 1, wherein the drug provides the patient with relief from allodynia.

3. A method for treating a patient suffering from allodynia, comprising administering to a patient in need of such treatment an effective amount of an agonist drug capable of binding to at least one peripheral neuronal nicotinic receptor but which does not readily cross the blood-brain barrier.

4. A method for treating a patient suffering from neuropathic pain, comprising administering to a patient in need of such treatment an effective amount of a quaternary nicotinic compound.

5. A method for treating a patient suffering from neuropathic pain by effecting peripheral neuronal nicotinic receptors at the level of the dorsal root ganglia, comprising administering to a patient in need of such treatment an effective amount of a nicotinic agonist compound that does not readily cross the blood-brain barrier.

6. A method for reducing side effects associated with agonist activation of central neuronal nicotinic receptors, comprising administering to a patient in need of such treatment an effective amount of a nicotinic agonist compound that does not readily cross the blood-brain barrier.
